(19) United States
(12) Patent Application Publication
(10) Pub. No.: US 2021/0206730 A1
(43) Pub. Date: Jul. 8, 2021

(54) PROCESS FOR PREPARING ISOCYANURATE

(71) Applicant: Evonik Operations GmbH, Essen (DE)

(72) Inventors: Emmanouil Spyrou, Schermbeck (DE); Holger Loesch, Herne (DE); Susanne Kreischer, Herten (DE); Andrea Diesveld, Gescher (DE); Andrea Thesing, Ahaus (DE)

(73) Assignee: Evonik Operations GmbH, Essen (DE)

(21) Appl. No.: 17/126,152

(22) Filed: Dec. 18, 2020

(30) Foreign Application Priority Data
Jan. 6, 2020 (EP) 20150329.9

(51) Int. Cl. C07D 251/34; B01J 31/02

(52) U.S. Cl. CPC C07D 251/34; B01J 31/0239

(57) ABSTRACT

The present invention provides a process for preparing isocyanurate from diisocyanate, in which i) prior to the reaction the peroxide content of the diisocyanate to be used is determined, and thereafter ii) a) if the determined peroxide content is greater than 10 mmol/kg, the diisocyanate is subjected to distillative purification until the determined peroxide content is less than or equal to 10 mmol/kg, orb) if the determined peroxide content is less than or equal to 10 mmol/kg, no further action is taken, and iii) distilled diisocyanate with respect to a) and/or untreated diisocyanate with respect to b) is subsequently converted to isocyanurate.

### CROSS REFERENCE TO RELATED APPLICATIONS

This application is a 35 U.S.C. § 119 patent application which claims the benefit of European Application No. 20150329.9 filed Jan. 6, 2020, which is incorporated herein by reference in its entirety.

### FIELD

The present invention relates to a process for preparing isocyanurates from diisocyanates.

Isocyanurates are valuable starting materials for the preparation of polyurethane paints. In this respect, processes for preparing them are likewise of great interest.

### BACKGROUND

EP 0 082 987 A2 discloses a process for preparing isocyanurates from mixtures containing MPDI and EBDI. The preparation is performed within 1-60 minutes at temperatures of from 40-140° C. in the presence of a catalyst which may preferably be a quaternary ammonium salt of an organic acid.

EP 1 170 283 A2 discloses a process for preparing low-odor and storage-stable isocyanurates in which IPDI is converted within 30 seconds to 2 hours at temperatures of from 0 to 200° C. in the presence of a catalyst which may be an ammonium salt of an acid.

EP 1 273 603 A1 discloses a process for preparing low-odor and storage-stable isocyanurates in which IPDI is converted within 3 minutes to 3 hours at temperatures of from 0-160° C. in the presence of a catalyst which contains at least one quaternary nitrogen atom and is based on a tricyclic diamine.

EP 1 454 933 A1 discloses a process for preparing low-odor and storage-stable isocyanurates in which IPDI is converted within 2-30 minutes at temperatures of from 20-120° C. and in a pressure range of from 0.5-5 bar in the presence of a catalyst which contains at least one quaternary nitrogen atom and is based on a tricyclic diamine, and subsequently the catalyst is thermally deactivated.

The known catalyst-employing processes for preparing isocyanurates have the disadvantage of resulting in products having excessively high color numbers. It is thus an object of the present invention to produce as little coloration as possible for a given degree of isocyanurate conversion.

### SUMMARY

Surprisingly, it has been found that peroxide contaminations are present in particular in older diisocyanate batches, which during the isocyanurate synthesis lead to the low degrees of conversion and high color numbers mentioned. It has furthermore been found that higher yields and lower color numbers result when there is a peroxide content of less than 10 mmol/kg in the diisocyanate batches used.

### DETAILED DESCRIPTION

The present invention accordingly provides a process for preparing isocyanurate from diisocyanate, in which
i) prior to the reaction the peroxide content of the diisocyanate to be used is determined, and thereafter
ii) a) if the determined peroxide content is greater than 10 mmol/kg, the diisocyanate is subjected to distillative purification until the determined peroxide content is less than or equal to 10 mmol/kg, or
   b) if the determined peroxide content is less than or equal to 10 mmol/kg, no further action is taken, and
iii) distilled diisocyanate with respect to a) and/or untreated diisocyanate with respect to b) is subsequently converted to isocyanurate.

The present invention provides a process for preparing isocyanurate from diisocyanate. The reactant “diisocyanate” may be a single diisocyanate or a mixture of diisocyanates. The reactant is preferably precisely one diisocyanate.

Preferably, at least one of the diisocyanates used is a (cyclo)aliphatic diisocyanate, that is to say a diisocyanate having at least one isocyanate group bonded directly to an aliphatic ring and possibly a further aliphatically bonded (that is to say joined to the aliphatic ring via an alkylene radical) isocyanate group. More preferably, only a single (cyclo)aliphatic diisocyanate is used. Very particularly preferably, at least one of the diisocyanates used is isophorone diisocyanate (IPDI) or 4,4′-diisocyanatodicyclohexylmethane (H12MDI). More preferably still, isophorone diisocyanate is used as the sole diisocyanate. If isophorone diisocyanate is used, it is unimportant whether it has been obtained via the urea process or via the phosgene process.

The product “isocyanurate” in principle relates to isocyanurate group-containing product mixtures comprising chain-like and crosslinked polyisocyanatoisocyanurates, triisocyanatomonoisocyanurates (“trimers”) and possibly precursors to the trimer formation.

The isocyanurate group-containing product mixture is preferably monomer-containing trimer preparable by partial trimerization of diisocyanate. The process for preparing isocyanurate according to the invention is thus preferably a process for the partial trimerization of diisocyanate in which essentially triisocyanatomonoisocyanurates and precursors of isocyanurates are produced.

Prior to the partial or complete conversion of diisocyanate to isocyanurate, the peroxide content in the diisocyanate to be used is determined. The peroxide content is determined according to DIN EN ISO 27 107 and is specified in mmol/kg.

“Prior” to the conversion is preferably understood in this case to mean a time window of from 14 days to 5 minutes before mixing reactant and catalyst. It is very particularly preferably understood to mean a point in time of 2 days prior to the conversion.

If only one diisocyanate is used to prepare isocyanurate, the peroxide content of the diisocyanate to be used is the content of peroxide in mmol based on the total mass of the diisocyanate to be used in kilograms. If more than one diisocyanate is used to prepare isocyanurate, the peroxide content of the diisocyanate to be used is the total content of peroxide in mmol based on the total mass of all diisocyanates to be used in kilograms.

If the peroxide content, determined as defined hereinabove, is less than or equal to 10 mmol/kg, no further action is taken, since appreciable disadvantages due to the presence of such a concentration of peroxide are not to be expected. The reactant(s) may thus be converted directly to isocyanurate.

However, if the peroxide content, determined as defined hereinabove, is greater than 10 mmol/kg, the reactant diisocyanate is subjected to distillative purification. If the intention is to prepare isocyanurate from just one diisocyanate, each batch of the diisocyanate having a peroxide content of greater than 10 mmol/kg is purified by distillation until the peroxide content of each batch is less than or equal to 10 mmol/kg. If the intention is to prepare isocyanurate from a plurality of different diisocyanates, each batch of each diisocyanate having a peroxide content of greater than 10 mmol/kg is purified by distillation until the peroxide content of each batch of each diisocyanate is less than or equal to 10 mmol/kg.

The distillative purification is preferably conducted in suitable distillation columns or distillation units, for example short-path or thin-film distillation apparatuses, at suitable pressures and temperatures depending on the boiling temperature of the diisocyanates. The minimum distillation temperature should in this case preferably not be below 100° C.

The conversion of diisocyanate to isocyanurate is preferably conducted in the presence of at least one catalyst at temperatures of from 0-160° C. The pressure is not set specially in this case and corresponds to ambient pressure, which is close to 1 bar. Preferred reaction temperatures are 40-140° C. and more preferably still 60-130° C.

Preferred reaction times are between 3 minutes and three hours.

Suitable catalysts may be selected from the group consisting of tertiary amines, alkali metal salts of carboxylic acids, quaternary ammonium salts, aminosilanes and quaternary hydroxyalkylammonium salts. Preferred catalysts are N-(2-hydroxypropyl)-N,N,N-trimethylammonium 2-ethylhexanoate (75% in diethylene glycol, available as DABCO TMR), or OH-containing quaternary ammonium compounds (available for example as EP BZ 7078 B from Evonik).

The catalyst is preferably used in amounts of from 0.05%-1.5% by weight, more preferably in amounts of from 0.1%-0.8% by weight, more preferably still 0.4%-0.7% by weight, based on the mass of diisocyanate used.

The reaction can optionally be conducted in the presence of at least one cocatalyst, at least one solvent and/or at least one auxiliary.

Preferred cocatalysts can be selected from the group consisting of OH-functionalized compounds and Mannich bases formed from secondary amines and aldehydes or ketones.

If a solvent is used, it is preferably used to dissolve the catalyst for the purpose of achieving a more exact metering and optimal mixing. Solvents are preferably selected from water, low molecular weight alcohols (especially methanol and ethylene glycol) and low molecular weight organic acids (especially acetic acid or hexanoic acid).

The process according to the invention can be carried out either batchwise or continuously. It is preferably carried out in a batch process.

Very particularly preferably, the process according to the invention is carried out as a process for the partial trimerization of diisocyanate, that is to say during the conversion of diisocyanate to isocyanurate trimer the aim is for a conversion of markedly below 100% (determined via the residual content of NCO groups), preferably between 20% and 80%, more preferably between 25% and 60%, more preferably still between 30% and 45%. The conversion is determined simply by way of a titrimetric determination of the NCO number according to DIN EN ISO 14896:2009-07, that is to say a sample is dissolved in a non-protic solvent (for example acetone or ethyl acetate), then an excess of dibutylamine is added and the unreacted fraction is back-titrated with 0.1% hydrochloric acid.

In such a process for the partial trimerization of diisocyanate, diisocyanate is left to react in the presence of the catalyst, optionally with use of solvents and/or auxiliaries, until the desired conversion has been attained. If the reaction does not terminate upon achieving the desired conversion, it may be terminated by deactivation of the catalyst. This can be done by adding a catalyst inhibitor such as for example p-toluenesulfonic acid, hydrogen chloride or dibutyl phosphate. However, a disadvantage here is a possibly undesired contamination of the resulting isocyanurate group-containing polyisocyanate with the catalyst inhibitor.

Surprisingly, it has moreover been found that in the case of peroxide contents of between 0.1 and 10 mmol/kg, the corresponding diisocyanates, especially IPDI, can be converted directly to product mixtures of a partial trimerization (preferably comprising monomeric IPDI, trimeric isophorone isocyanurate and higher oligomers with isocyanurate structure) having good properties, without termination of the reaction using a disadvantageous catalyst inhibitor being necessary.

The present invention accordingly thus also provides a process for preparing isocyanurate from diisocyanate, in which
i) prior to the reaction the peroxide content of the diisocyanate to be used is determined, and thereafter
ii) a) if the determined peroxide content is greater than 10 mmol/kg, the diisocyanate is subjected to distillative purification until the determined peroxide content is less than or equal to 10 mmol/kg but greater than or equal to 0.1 mmol/kg, or
   if the determined peroxide content is less than 0.1 mmol/kg, peroxide is added until the peroxide content is greater than or equal to 0.1 mmol/kg but less than or equal to 10 mmol/kg, or
   b) if the determined peroxide content is less than or equal to 10 mmol/kg but greater than 0.1 mmol/kg, no further action is taken, and
iii) distilled diisocyanate with respect to a) and/or untreated diisocyanate with respect to b) is subsequently converted to isocyanurate.

### Examples

100 g of isophorone diisocyanate are in each case heated to 100° C. and admixed with 0.5% of a trimerization catalyst (DABCO TMR, Air Products, or Vestanat EP BZ 7078 B, Evonik). The mixture heats up as a result of an exothermic reaction to a temperature of below 160° C. and is then cooled. The residual NCO content is determined and with it the conversion and the color number.

The starting material used is either isophorone diisocyanate obtained by the urea process (IPDI U) or by the phosgene process (IPDI P).

The following observations are made: The more peroxide the IPDI contains, the lower the conversion/the reactivity. The color is associated firstly with the peroxide content but also secondly with the conversion. That is to say, peroxides always lead to lower conversion and also usually lead to higher color numbers. This applies both to IPDI (U) and to IPDI (P), and is applicable for both catalysts used as well.
a) IPDI U, catalyst: 0.5% by weight of DABCO TMR

| | | | | | | | | Color/ |
|---|---|---|---|---|---|---|---|---|
| | Peroxide | | | | NCO | | Color number | Conversion |
| No. | content | T_{Start} | T_{End} | | content | Conversion | Hazen/Gardner | [Hz/%] |
| 1 | <0.1 | 100° C. | 136.9° | C. | 30.60% | 38.40% | 228/1.0 | 6 |
| 2 | 19 | 100° C. | 120.6° | C. | 32.40% | 28% | 747/4.0 | 27 |
| 3 | 32 | 100° C. | 122.4° | C. | 32.90% | 25.90% | 730/3.9 | 28 |
| 4 | 32 | 100° C. | 111.3° | C. | 32.50% | 28.00% | 915/4.4 | 33 |
| 5 | 56 | 100° C. | 102° | C. | 35.20% | 13.80% | 390/2.3 | 28 |

b) IPDI P, catalyst: 0.5% by weight of BZ 7078

| | | | | | | | Color/ |
|---|---|---|---|---|---|---|---|
| | Peroxide | | | NCO | | Color number | Conversion |
| No. | content | T_{Start} | T_{End} | content | Conversion | Hazen/Gardner | [Hz/%] |
| 1 | <0.1 | 100° C. | 134.7° C. | 29.70% | 42.90% | 514/3.0 | 12 |
| 2 | 13.5 | 100° C. | 116.2° C. | 32.70% | 27.00% | 567/3.3 | 21 |
| 3 | 23 | 100° C. | 101.4° C. | 35.60% | 11.60% | 396/2.3 | 34 |

1. A process for preparing an isocyanurate from diisocyanate, in which
i) determining the peroxide content of the diisocyanate prior to the reaction, determined peroxide content, and thereafter
ii) a) if the determined peroxide content is greater than 10 mmol/kg, the diisocyanate is subjected to distillative purification until the determined peroxide content is less than or equal to 10 mmol/kg, or
b) if the determined peroxide content is less than or equal to 10 mmol/kg, no further action is taken, and
iii) converting the distilled diisocyanate with respect to a) and/or untreated diisocyanate with respect to b) to isocyanurate.

2. The process according to claim 1, wherein
the process is a process for preparing isocyanurate from a diisocyanate.

3. The process according to claim 1, wherein
at least one of the diisocyanates used is a (cyclo)aliphatic diisocyanate.

4. The process according to claim 1, wherein
at least one of the diisocyanates used is isophorone diisocyanate or 4,4′-diisocyanatodicyclohexylmethane.

5. The process according to claim 1, wherein
the conversion of diisocyanate to isocyanurate is conducted in the presence of at least one catalyst at temperatures of from 0 to 160° C.

6. The process according to claim 5, wherein
the catalyst is selected from the group consisting of N-(2-hydroxypropyl)-N,N,N-trimethylammonium 2-ethylhexanoate and OH-containing quaternary ammonium compounds.

7. The process according to claim 1, wherein
the conversion of diisocyanate to isocyanurate trimer is between 20% and 80% by weight.

8. The process according to claim 1, wherein in step
ii) a) if the determined peroxide content is greater than 10 mmol/kg, the diisocyanate is subjected to distillative purification until the determined peroxide content is less than or equal to 10 mmol/kg but greater than or equal to 0.1 mmol/kg,
or
if the determined peroxide content is less than 0.1 mmol/kg, peroxide is added until the peroxide content is greater than or equal to 0.1 mmol/kg but less than or equal to 10 mmol/kg, or
b) if the determined peroxide content is less than or equal to 10 mmol/kg but greater than 0.1 mmol/kg, no further action is taken.

9. The process according to claim 2, wherein
at least one of the diisocyanates used is a (cyclo)aliphatic diisocyanate.

10. The process according to claim 2, wherein
at least one of the diisocyanates used is isophorone diisocyanate or 4,4′-diisocyanatodicyclohexylmethane.

11. The process according to claim 2, wherein
the conversion of diisocyanate to isocyanurate is conducted in the presence of at least one catalyst at temperatures of from 0 to 160° C.

12. The process according to claim 11, wherein
the catalyst is selected from the group consisting of N-(2-hydroxypropyl)-N,N,N-trimethylammonium 2-ethylhexanoate and OH-containing quaternary ammonium compounds.

13. The process according to claim 3, wherein
the conversion of diisocyanate to isocyanurate is conducted in the presence of at least one catalyst at temperatures of from 0 to 160° C.

14. The process according to claim 13, wherein
the catalyst is selected from the group consisting of N-(2-hydroxypropyl)-N,N,N-trimethylammonium 2-ethylhexanoate and OH-containing quaternary ammonium compounds.

15. The process according to claim 4, wherein
the conversion of diisocyanate to isocyanurate is conducted in the presence of at least one catalyst at temperatures of from 0 to 160° C.

16. The process according to claim 15, wherein
the catalyst is selected from the group consisting of N-(2-hydroxypropyl)-N,N,N-trimethylammonium 2-ethylhexanoate and OH-containing quaternary ammonium compounds.

17. The process according to claim 2, wherein
the conversion of diisocyanate to isocyanurate trimer is between 20% and 80% by weight.

18. The process according to claim 2, wherein in step
ii) a) if the determined peroxide content is greater than 10 mmol/kg, the diisocyanate is subjected to distillative purification until the determined peroxide content is less than or equal to 10 mmol/kg but greater than or equal to 0.1 mmol/kg,
or
if the determined peroxide content is less than 0.1 mmol/kg, peroxide is added until the peroxide content is greater than or equal to 0.1 mmol/kg but less than or equal to 10 mmol/kg, or
b) if the determined peroxide content is less than or equal to 10 mmol/kg but greater than 0.1 mmol/kg, no further action is taken.

19. The process according to claim 3, wherein
the conversion of diisocyanate to isocyanurate trimer is between 20% and 80% by weight.

20. The process according to claim 3, wherein in step
ii) a) if the determined peroxide content is greater than 10 mmol/kg, the diisocyanate is subjected to distillative purification until the determined peroxide content is less than or equal to 10 mmol/kg but greater than or equal to 0.1 mmol/kg,
or
if the determined peroxide content is less than 0.1 mmol/kg, peroxide is added until the peroxide content is greater than or equal to 0.1 mmol/kg but less than or equal to 10 mmol/kg, or
b) if the determined peroxide content is less than or equal to 10 mmol/kg but greater than 0.1 mmol/kg, no further action is taken.
